(19) United States
(12) Patent Application Publication
(10) Pub. No.: US 2013/0133424 A1
(43) Pub. Date: May 30, 2013

(54) SYSTEM-BASED MOTION DETECTION

(71) Applicant: Thomas Alan Donaldson, London (GB)

(72) Inventor: Thomas Alan Donaldson, London (GB)

(73) Assignee: AliphCom

(21) Appl. No.: 13/528,830

(22) Filed: Jun. 20, 2012

Related U.S. Application Data
(63) Continuation-in-part of application No. 13/158,372, filed on Jun. 10, 2011.
(60) Provisional application No. 61/499,476, filed on Jun. 21, 2011.

(51) Int. Cl. G01P 15/00; A61B 5/024

(52) U.S. Cl. CPC G01P 15/00; A61B 5/02444
USPC 73/510; 600/508

(57) ABSTRACT

Techniques for system-based motion detection is described, including a first accelerometer configured to detect a first acceleration associated with a system element, a second accelerometer configured to detect a second acceleration associated with the system, and a differential amplifier configured to generate a signal corresponding to the first acceleration, wherein the signal is used to distinguish the first acceleration from the second acceleration.

### CROSS-REFERENCE TO RELATED APPLICATIONS

This patent application is a continuation-in-part of U.S. Non-Provisional patent application Ser. No. 13/158,372, filed Jun. 10, 2011, and claims the benefit of U.S. Provisional Patent Application No. 61/499,476, filed Jun. 21, 2011, both of which are herein incorporated by reference for all purposes.

### FIELD

The present invention relates generally to electrical and electronic hardware, computer software, wired and wireless network communications, and wearable computing devices. More specifically, techniques for system-based motion detection are described.

### BACKGROUND

Accelerometers have proven to be a useful device for detecting motion as they are relatively small, relatively low cost, and consume relatively low power. As a result of these advantages, systems have been developed that use accelerometers for the detection of motion. For example, accelerometers are used in “smartphones” to detect the orientation and movement of the device. This may be done as part of determining whether to display the screen in portrait or landscape mode, to assist in implementing certain user interface functions or elements (for example scrolling or navigating), or to detect specific motions characteristic of a potential problem (for example, when the phone is dropped). In such examples, the accelerometer is used to measure the absolute acceleration of the system (in this example, the smartphone) as a whole.

Accelerometers have proven useful at measuring the acceleration (and therefore the orientation and motion) of a system as a whole, because their measurement method is performed in terms of an absolute frame of reference (i.e., the world). This is as opposed to a relative frame of reference such as the casing of a device.

However, this reliance on a measurement based on an absolute frame of reference can be a disadvantage when using an accelerometer to detect the relative motion of an element that is part of a system (i.e., the motion of a system element relative to one or more other elements of the system). This is because the accelerometer may respond to accelerations of the system as a whole as well as to accelerations of the sub-system of interest, and in many cases, the motion of the system as a whole may be larger than the motion of the sub-system. In many systems, it can be a very complex problem to distinguish movements of the system as a whole from movements of a sub-system of interest based on their combined accelerations.

As a conventional solution where this disadvantage may become evident, consider the problem of detecting movements of certain parts of the body. In conventional solutions, an accelerometer, while detecting the movement of the body part of interest, also detects the movement of the body as a whole, or the motion of other parts of the body to which the part of interest is connected (or with which it is closely arranged). In other words, in conventional solutions, an accelerometer used to provide an input to a computer system by detecting hand motion (by, for example, being attached near the wrist or a finger) is also likely to be confused (i.e., to generate spurious signals) by a user walking, thus requiring a user to remain relatively still for the duration of an input session; an accelerometer used to detect voice activity (that is, whether or not a user is speaking) by movement of the skin of the cheek or by detecting vibrations conducted through bone is likely to be confused (i.e., to generate spurious signals) by a user walking, moving their head, or performing other motion, and may require significantly increased signal processing capabilities to reliably and accurately detect actual voice activity; or an accelerometer used to detect the pulse at the wrist of a user and is often confused by movement of the wrist or by user motion (e.g., walking, running, bending, twisting, or other types of movement) so that the pulse cannot be reliably detected.

Thus, what is needed is a solution for system-based motion detection without the limitations of conventional techniques

### SUMMARY

Various techniques (i.e., examples, which may be used interchangeably with “embodiments”) are directed to systems, apparatuses, devices, and methods for using accelerometers or other devices capable of detecting motion to detect the motion of an element or part of an overall system. Techniques may be used to accurately and reliably detect the motion of a part of the human body or an element of another complex system while avoiding the limitations or disadvantages of currently known methods of making such measurements.

In some examples, techniques described include a first and second accelerometer, where the first accelerometer is coupled and configured to receive signals from a system element of interest as opposed to an entire system, which may be distinguished from the implementation of a second accelerometer, where both accelerometers are coupled to the system as a whole. A differential amplifier or element capable of similar functions may be used to generate a signal corresponding to an acceleration experienced by the system element, where this acceleration is independent of that experienced by the system as a whole.

### BRIEF DESCRIPTION OF THE DRAWINGS

FIG. 1 is an exemplary schematic illustration of the primary elements or components;

FIG. 2 is an exemplary cross-sectional diagram illustrating a human wrist based pulse detection system;

FIG. 3 illustrates an exemplary mechanism for providing a coupling between a mounting element and a first and second accelerometer;

FIG. 4 illustrates an exemplary application of system-based motion detection for detecting speech;

FIG. 5 illustrates an exemplary data-capable strapband configured to perform system-based motion detection;

FIG. 6 is another illustration of an exemplary data-capable strapband configured to perform system-based motion detection; and

FIG. 7 is a further illustration of an exemplary data-capable strapband configured to perform system-based motion detection.

### DETAILED DESCRIPTION

Various techniques described are directed to systems, apparatuses, devices, and methods for the detection of motion, where the motion may be the result of an applied force or impulse (and hence may result in one or more of an acceleration, a velocity, or a displacement of a system element). In some embodiments, the motion may be detected using an accelerometer which responds to an applied force and produces an output signal representative of the acceleration (and hence in some cases a velocity or displacement) produced by the force. Embodiments may be used to detect the motion of a sub-component of a system while the system itself is undergoing motion in a manner that is not predictable or in some cases is not known to the motion measurement or detection system. In some embodiments, the described techniques may use an accelerometer to detect the motion of a part of a human body while that body may be moving in an otherwise unpredictable manner.

Techniques described are directed to systems, apparatuses, devices, and methods for using accelerometers or other devices capable of detecting motion to detect the motion of an element or part of an overall system. In some examples, the described techniques may be used to accurately and reliably detect the motion of a part of the human body or an element of another complex system while avoiding the limitations or disadvantages of currently known methods of making such measurements.

In some examples, described techniques may include a first and second accelerometer, where the first accelerometer is more strongly coupled to the system element of interest than the second accelerometer, and where both accelerometers are coupled to the system as a whole (or to an aspect of the system that undergoes motion representative of the system as a whole). A differential amplifier or element capable of similar functions may be used to generate a signal corresponding to an acceleration experienced by the system element, where this acceleration is independent of that experienced by the system as a whole.

In some examples; the following elements may be implemented, including two (2) closely-matched accelerometers (i.e., “closely-matched” may include being sufficiently similar in signal response to an identical or substantially identical stimulus such that the difference in those signal responses is insignificant when compared to the individual signal responses to the original stimulus, where the level of significance is determined by an application such as a computer program, software, firmware, or other logic coupled or in data communication with the accelerometers). For example, in an application where a dynamic range of 20 dB is used, the difference in accelerometer responses may be less than −20 dB. In other examples, the difference in accelerometer responses may be more or less than −20 db. As described, a matched coupling system may be disposed (i.e., placed, positioned, configured, or otherwise implemented, structurally and/or functionally) between each of the accelerometers and a system housing, with coupling systems being sufficiently similar in their responses to an identical stimulus that the difference in those responses may be insignificant compared to the individual responses of each accelerometer to that stimulus. Further, a coupling system may be disposed between one of the accelerometers and the system element that undergoes motion to generate a signal of interest. Still further, a decoupling system (which may be implemented, in some examples, in the form of a lack of coupling) may be disposed between one of the accelerometers and a system element (e.g., an electrical, electronic, mechanical, or electro-mechanical element of a system in which the two or more accelerometers are implemented) and configured to generate a signal of interest that is used to determine a difference between accelerations detected by the accelerometers. In other examples, a differential mode signal determination element may also be implemented, optionally together with a common mode signal determination element.

In some examples, two accelerometers may be provided, including one coupled to a system element that undergoes motion to generate a signal of interest (i.e., an accelerometer moves with the system element to a degree sufficient that the motion of the accelerometer, as reflected by the signal generated by the accelerometer) can be used to represent the motion of the system element to a degree or accuracy appropriate to the application), and a second accelerometer that is not configured to detect accelerations from the system element as opposed to the system itself (i.e., “poorly” coupled), and with both being equally coupled to the system as a whole. In other examples, one or more accelerometers may be configured to output data or signals in a differential mode.

In some examples, a heart rate, pulse and/or blood pressure at a user's wrist may be detected by means of two accelerometers, with one being well coupled to a radial artery, and a second one being poorly coupled, with both being well coupled to a wrist as a whole.

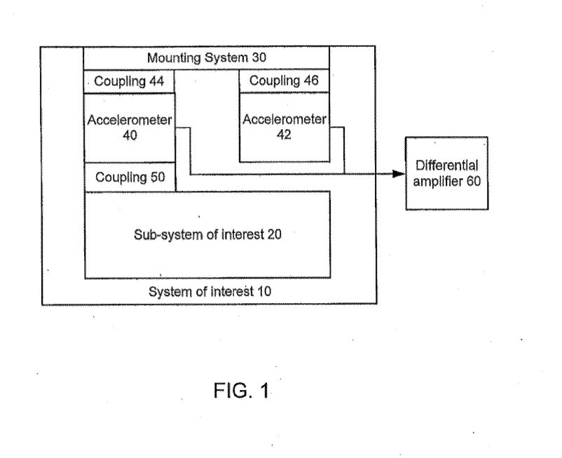

FIG. 1 is an exemplary schematic illustration of the primary elements or components. These elements or components include a system of interest 10, a sub-system whose movement relative to the system 10 as a whole is of interest 20, a mounting system 30 coupled to the system as a whole, a first 40 and a second 42 accelerometer, with each being well (i.e., relatively strongly compared to a weakly coupled element) coupled to the mounting system (as indicated by coupling elements 44 and 46 in the figure and a coupling system 50 serving to couple one of the accelerometers 40 to the sub-system 20 whose motion is of interest. In some examples, a 3-way differential amplifier 60 may be included, to which each of the 3 outputs (i.e., the signals corresponding to the X, Y, and Z components of acceleration or motion) of each of the accelerometers is connected (where it is assumed that accelerometers 40 and 42 are of a type that provide an analog electrical signal as an output). In the case where the accelerometers provide a digital electrical signal as an output (for example as data transmitted over an I2C interface), an appropriate equivalent differential signal determining means (for example, microcode performing a subtraction of one signal from the other, running on a microcontroller) may be used.

Note that a wide range of suitable mounting systems are known, including for example a circular band kept under tension, screws, nails or glue fixing a casing to the system of interest, and so on.

In some examples, a coupling system (as represented by elements 44 and 46) is intended to be effective to ensure that motions of the mounting system 30 cause motion of the accelerometers sufficiently similar to the motion of the mounting, at least for motions within a range of interest (i.e., the velocities, accelerations, vibrational frequencies, etc. expected to be encountered in typical operation of the overall system and its component sub-systems), while also allowing for motions of the accelerometers that may not be due to the motion of the mounting. Note that a range of suitable coupling systems are known, including, for example a spring, a bushing, O rings, gaskets, and the like, with such coupling systems or devices being made from metal, rubber, plastic and so on.

In general, the accelerometers are effective for measuring their own acceleration and in response providing an electrical or electronic output signal that is proportional to the measured acceleration. It may be understood that a number of suitable accelerometers are available, from companies including Bosch, ST Microelectronics and so on, with the accelerometers providing one, two or three axes of acceleration data at different sample rates, and delivering data in analog or digital electronic form via a variety of interfaces (such as analog wire, I2C digital interface, SP1 digital interface, GPIO-based digital interface, and so on). The use and interconnection of such interfaces is believed to be well known to those skilled in the art.

Differential amplifier 60 operates to provide an output signal whose value is approximated by the difference between the values of the inputs (that is the two accelerometer readings along some axis) multiplied by some constant K (where this relationship typically holds within a specified sampling frequency range, where such a frequency range overlaps the frequency range of motions of interest). As a result of the above-described configuration, an output of the differential amplifier is an electrical or data signal whose amplitude is substantially proportional to the motion of the subsystem of interest and only poorly related to the motion of the system as a whole.

Note that a variety of elements or devices that may be configured to operate as a differential amplifier are available, including an operational amplifier in a differential configuration (as is well known to those skilled in the art), or a microprocessor or microcontroller configured to perform the subtraction of one signal from another, a digital signal processor running an algorithm to calculate the difference between the signals, etc.

Note also that while a simple difference between the two accelerometer signals is sufficient to produce a differential signal, in some embodiments a more optimal differential signal (i.e., one in which signals caused by the common-mode movements of the system as a whole are maximally removed) may be produced by more advanced processing techniques. For example, a digital signal processor may apply a calibration transform on the signals prior to taking their difference, so as to minimize the effects of any differences in the response of the signal.

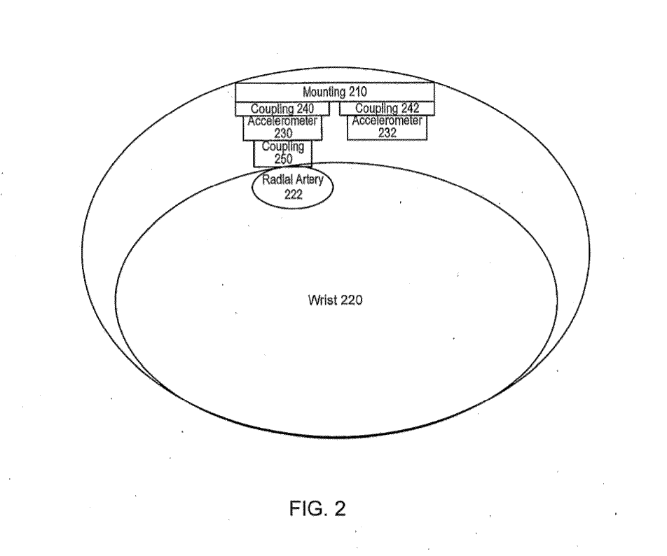

FIG. 2 is an exemplary cross-sectional diagram illustrating a human wrist based pulse detection system. A mounting system 210 encircles the wrist 220 (in which is shown an approximate position of the radial artery 222). A first accelerometer 230 and a second accelerometer 232 are shown, each coupled to the mounting system in a similar manner (as represented by coupling elements 240 and 242 in the figure). Note that as shown in the figure, the two accelerometers are mounted relatively close together. As the system as a whole moves (and in particular rotates) accelerometers that are not sufficiently close together may not receive an identical signal from the system as a whole, and this difference may remain in the differential mode Signal. Consider for example the case of a rotating system where one accelerometer is mounted on the axis of rotation and the other near the periphery of the device on the sub-system of interest. In this instance the acceleration on each of the two accelerometers is considerably different (the one on the axis of rotation being subject to little or no acceleration from the rotation). In general, it is advantageous for the accelerometers to be mounted as close as possible to each other, and in any case sufficiently close that differences in the signal caused by the motion of the system as a whole are insignificant (for the application of the device) compared to the signals caused by the motion of the sub-system of interest. The first accelerometer 230 is coupled to the wrist above the radial artery 222 by means of a coupling element 250. Note that the second accelerometer 232 is not coupled to the wrist except via coupling 242 and mounting system 210.

Mounting system 210 may comprise, for example, a band encircling the wrist in a manner so as to limit movement of the band relative to the wrist. For example, the band might be a metal, rubber, leather, or similar band sufficiently tight so that friction with the wrist prevents excessive movement relative to the wrist.

Accelerometers 230 and 232 may be of any suitable design or structure, such as the BMA150, BMA180 or similar devices by Bosch, or similar devices by other providers. Such devices are typically mounted on a printed circuit board, and are provided with power from a battery or other source (which is not shown in the figure).

Accelerometers 230 and 232 may be attached to mounting system 210 (e.g., the aforementioned band) by means of screws attaching their respective printed circuit board to the band, or by another suitable attachment mechanism. In such an embodiment, the printed circuit board(s) form the coupling elements 240 and 242 between accelerometers 230 and 232 and mounting system 210, as described in further detail.

Accelerometer 230 is coupled to radial artery 222 via the skin by means of coupling element 250, which in some embodiments may take the form of a hard rubber bushing. Accelerometer 230 is placed above radial artery 222, while the second accelerometer 232 is relatively poorly coupled to radial artery 222, being placed to the side and having an air gap between the skin and the printed circuit board on which the second accelerometer is mounted.

A microprocessor, for example, may be an ARM Cortex M3 microprocessor, and configured to function as a differential amplifier element (e.g., element 60 of FIG. 1). It may be connected to the two accelerometers via an I2C interface and receive data from each, and may be configured to provide differential output data by subtracting signals (i.e., the X, Y, and Z component signals) for one accelerometer from corresponding signals of another accelerometer. Note that a differential amplifier element may also be programmed to execute a set of instructions in order to operate as described. In other cases firmware or hardware may be used to implement the desired functions or operations.

Note that in the embodiment described with reference to FIG. 2, if the user moves their wrist, both accelerometers, being coupled to the band, which is tight around the wrist, may move with the wrist. Thus both accelerometers may register a similar response signal in response to wrist movements. Wrist movement may therefore be viewed as a ‘common-mode’ signal, as subtracting one signal from the other leaves very little remaining signal, while averaging the two signals gives a good estimate of the movement of the wrist.

As described herein, blood may flow through a radial artery creating a pulse, where the radial artery can expand to accommodate increased blood flow, and in so doing, may push against a bushing (or other coupling element, as represented by coupling 250). When pressed upon by coupling 250, a force may be applied and therefore an acceleration is created and detected by accelerometer 230 attached to the bushing (or other coupling element). This accelerometer may therefore be configured to generate a signal in response to a pulse flowing through a radial artery. Further, blood flow through a radial artery may be transmitted directly to a second accelerometer, as this accelerometer may be further away from the radial artery and does not come into contact or connection via any material able to transmit the force.

In some examples, a force applied to the first accelerometer is damped by the coupling attaching that accelerometer to the mounting system and further damped by the coupling attaching the mounting system to the second accelerometer. Therefore the force indirectly applied to the second accelerometer via the first accelerometer and the mounting system is substantially less than the force applied to the first accelerometer.

In some examples, a signal generated as a response to a pulse in the radial artery by the first accelerometer may be substantially larger than the signal generated in response by the second accelerometer. Arterial pulses may therefore be viewed as a ‘differential-mode’ signal, as subtracting one signal from the other (assuming equal gain for each signal) gives a relatively strong signal in response.

In some examples, the action of a differential amplifier or similar element (whether analog or digital) may be characterized as the removal of common-mode signals (in this case, the wrist movement) from differential-mode signals (in this case, pulse originated movement) and is effective even where the differential mode signal is many orders of magnitude less than the common-mode signal. Thus, the inventive elements act to significantly attenuate the appearance of wrist movement signals compared to pulse originated signals.

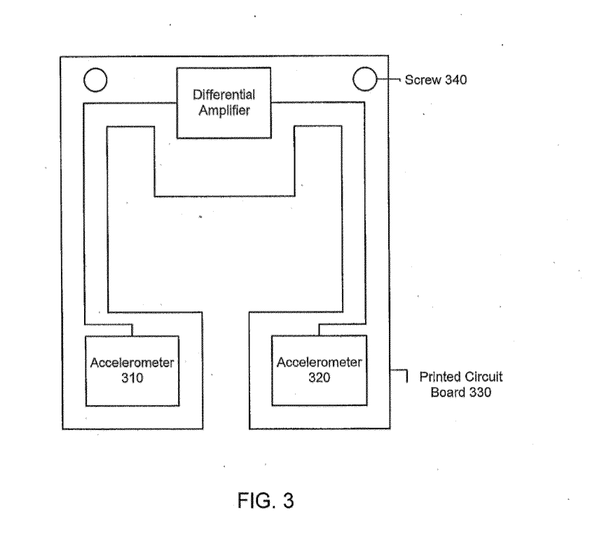

FIG. 3 illustrates an exemplary mechanism for providing a coupling between a mounting element and a first and second accelerometer. Here, FIG. 3 illustrates an example of a mechanism for providing a coupling between a mounting element and a first accelerometer 310 and a second accelerometer 320. In some examples, printed circuit board 330 on which the accelerometers are mounted is designed so that the accelerometers are each separated from the main bulk of the printed circuit board by a thin strip of printed circuit board which forms a somewhat flexible beam, allowing some movement of the accelerometer relative to the rest of the board. The main body of the printed circuit board may be mounted to the mounting system via screws 340 (or another suitable attachment mechanism) positioned near the end of the two beams.

In some examples, the flexibility of a beam may allow for acceleration-based (e.g., gravitational) forces on one or more accelerometers to cause a significant response, but, together with mounting hardware (e.g., screws, bolts, and the like) may cause very little force to be transmitted to the remaining accelerometer. Note also that movement of a mounting system may cause very similar movement in both accelerometers. This arrangement may be used to isolate one accelerometer from another, while providing an approximately equal coupling of an accelerometer to a common mounting element.

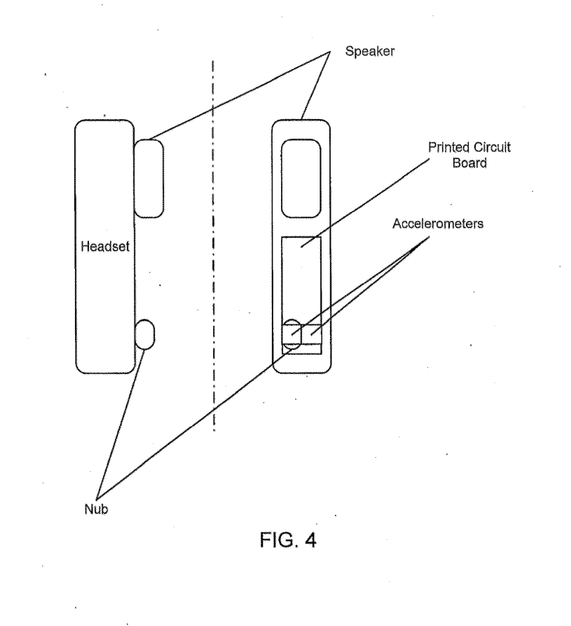

FIG. 4 illustrates an exemplary application of system-based motion detection for detecting speech. In some examples, described techniques may be used to implement Voice Activity Detection, which, as an example, may be used in telephony for improving detected speech (i.e., voice or acoustic signal) quality and reducing transmission bandwidth of speech signals, among many other uses. In some examples, a headset may be implemented as a stereo or mono Bluetooth headset. As described, the techniques provided herein may also be applied to stereo headsets that might be wired or used for reception of signals received using various types of wired and wireless media (e.g., WiFi or other RF or transmitted signals propagated using wired or wireless transmission media. At the front of a headset, facing the user's face, two accelerometers are provided. One is coupled to the face by means of a rubber ‘nub’ while the other is not coupled to the face. Both are attached to the headset via the PCB, which may be arranged as discussed above.

Note that head movements may affect each accelerometer equivalently. Movements of the cheek and jaw, and sound vibrations through the jawbone and cheek may affect an accelerometer in contact with a facial feature (e.g., a jaw or cheek), but may also be configured to not affect another accelerometer, and thus appear as a differential mode signal.

Therefore, this embodiment is capable of acting as a Voice Activity Detection system without being significantly susceptible to head movements. Such a system, constructed with one, two, or more accelerometers, may also be configured to vary in size, function, placement, or other aspects, including power consumption that may be lower than that of conventional solutions (e.g., microphones).

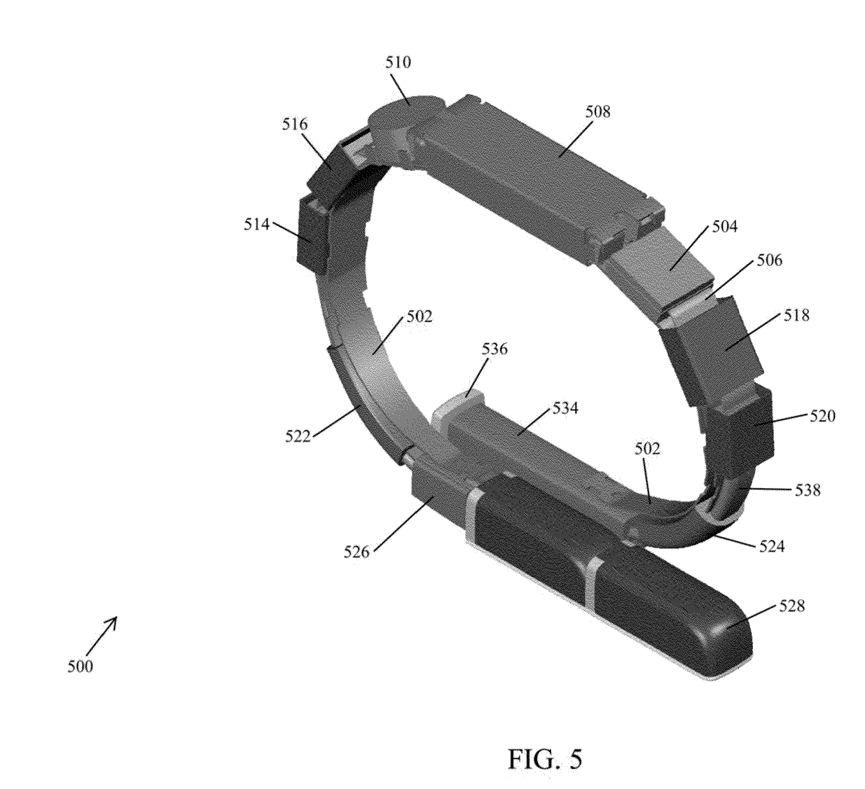

FIG. 5 illustrates an exemplary data-capable strapband configured to perform system-based motion detection. Here, band 500 includes framework 502, covering 504, flexible circuit 506, covering 508, motor 510, coverings 514-524, analog audio plug 526, accessory 528, control housing 534, control 536, and flexible circuit 538. In some examples, band 500 is shown with various elements (i.e., covering 504, flexible circuit 506, covering 508, motor 510, coverings 514-524, analog audio plug 526, accessory 528, control housing 534, control 536, and flexible circuit 538) coupled to framework 502. Coverings 508, 514-524 and control housing 534 may be configured to protect various types of elements, which may be electrical, electronic, mechanical, structural, or of another type, without limitation. For example, covering 508 may be used to protect a battery and power management module from protective material formed around band 500 during an injection molding operation. As another example, housing 504 may be used to protect a printed circuit board assembly (“PCBA”) from similar damage. Further, control housing 534 may be used to protect various types of user interfaces (e.g., switches, buttons, lights, light-emitting diodes, or other control features and functionality), one or more accelerometers, or other systems or systems elements configured to perform system-based motion detection from damage. In other examples, the elements shown may be varied in quantity, type, manufacturer, specification, function, structure, or other aspects in order to provide data capture, communication, analysis, usage, and other capabilities to band 500, which may be worn by a user around a wrist, arm, leg, ankle, heck or other protrusion or aperture, without restriction.

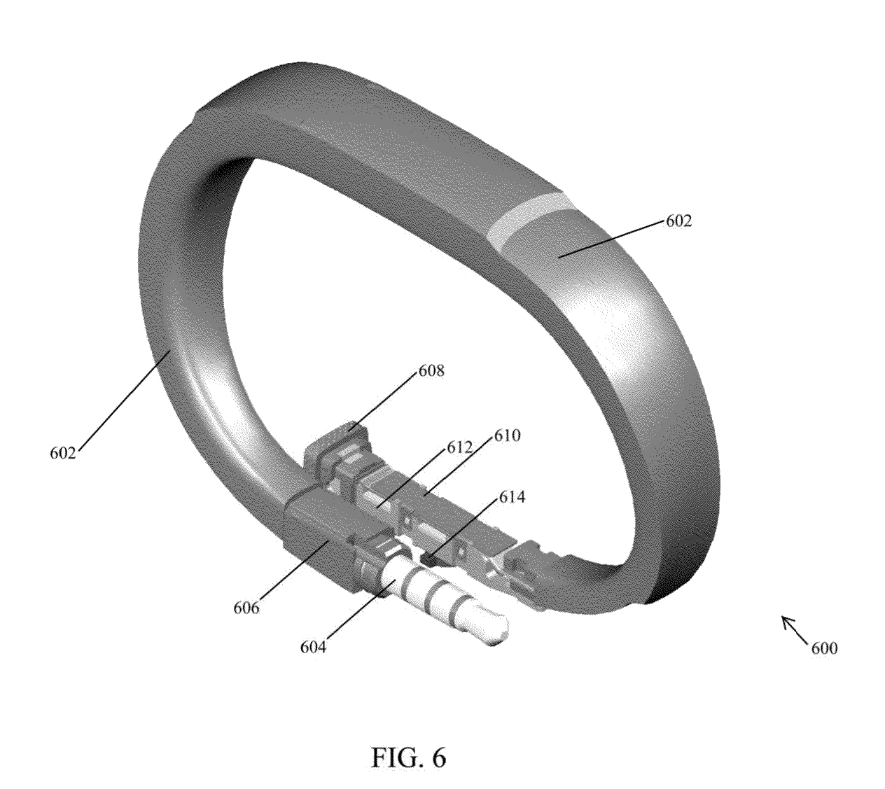

FIG. 6 is another illustration of an exemplary data-capable strapband configured to perform system-based motion detection. Here, band 600 includes molding 602, analog audio plug (hereafter “plug”) 604, plug housing 606, button 608, framework 610, control housing 612, and indicator light 614. In some examples, a first protective overmolding (i.e., molding 602) has been applied over band 500 (FIG. 5) and the above-described elements (e.g., covering 504, flexible circuit 506, covering 508, motor 510, coverings 514-524, analog audio plug 526, accessory 528, control housing 534, control 536, and flexible circuit 538) leaving some elements partially exposed (e.g., plug 604, plug housing 606, button 608, framework 610, control housing 612, and indicator light 614). However, internal PCBAs, flexible connectors, circuitry, one or more accelerometers, or other systems or systems elements configured to perform system-based motion detection, and other sensitive elements have been protectively covered with a first or inner molding that can be configured to further protect band 600 from subsequent moldings formed over band 600 using the above-described techniques. In other examples, the type, configuration, location, shape, design, layout, or other aspects of band 600 may be varied and are not limited to those shown and described. For example, plug 604 may be removed if a wireless communication facility is instead attached to framework 610, thus having a transceiver, logic, and antenna instead being protected by molding 602. As another example, button 608 may be removed and replaced by another control mechanism (e.g., an accelerometer that provides motion data to a processor that, using firmware and/or an application, can identify and resolve different types of motion that band 600 is undergoing), thus enabling molding 602 to be extended more fully, if not completely, over band 600. In yet other examples, molding 602 may be shaped or formed differently and is not intended to be limited to the specific examples shown and described for purposes of illustration.

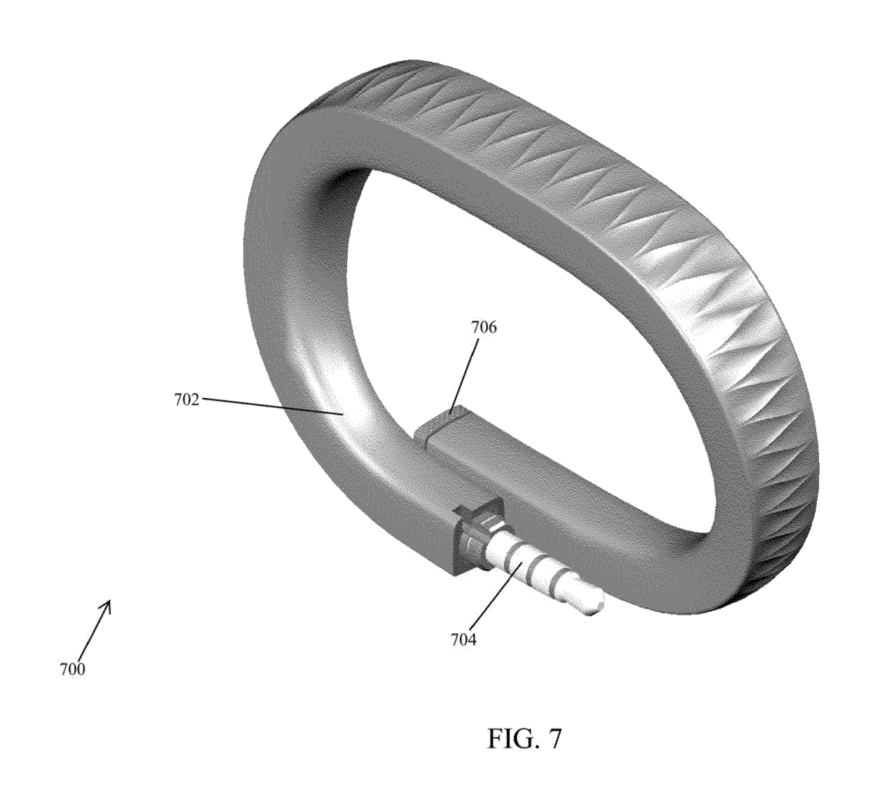

FIG. 7 is a further illustration of an exemplary data-capable strapband configured to perform system-based motion detection. Here, band 700 includes molding 702, plug 704, and button 706. As shown another overmolding or protective material has been formed by injection molding, for example, molding 702 over band 700. As another molding or covering layer, molding 702 may also be configured to receive surface designs, raised textures, or patterns, which may be used to add to the commercial appeal of band 700. In some examples, band 700 may be illustrative of a finished data-capable strapband (i.e., band 500 (FIG. 5), 600 (FIG. 6) or 700) that may be configured using one or more accelerometers, or other systems or systems elements configured to perform system-based motion detection to provide a wide range of electrical, electronic, mechanical, structural, photonic, or other capabilities.

Here, band 700 may be configured to perform data communication with one or more other data-capable devices (e.g., other bands, computers, networked computers, clients, servers, peers, and the like) using wired or wireless features. For example, a TRRS-type analog audio plug may be used (e.g., plug 704), in connection with firmware and software that allow for the transmission of audio tones to send or receive encoded data, which may be performed using a variety of encoded waveforms and protocols, without limitation. In other examples, plug 704 may be removed and instead replaced with a wireless communication facility that is protected by molding 702. If using a wireless communication facility and protocol, band 700 may communicate with other data-capable devices such as cell phones, smart phones, computers (e.g., desktop, laptop, notebook, tablet, and the like), computing networks and clouds, and other types of data-capable devices, without limitation. In still other examples, band 700 and the elements described above in connection with FIGS. 1-7, may be varied in type, configuration, function, structure, or other aspects, without limitation to any of the examples shown and described.

Although the foregoing examples have been described in some detail for purposes of clarity of understanding, the above-described inventive techniques are not limited to the details provided. There are many alternative ways of implementing the above-described inventive techniques. The disclosed examples are illustrative and not restrictive.

What is claimed:

1. A system, comprising:
a first element configured to generate an output signal representative of an acceleration applied to the first element;
a coupling element configured to couple the first element to a system element;
a second element configured to generate another output signal representative of another acceleration applied to the second element; and
a mounting element to which the first element and the second element are coupled, the mounting element coupled to the system.

2. The system of claim 1, wherein the first element is an accelerometer coupled to a system element.

3. The system of claim 1, wherein the first element is configured to detect the acceleration associated with the system element.

4. The system of claim 1, wherein the second element is coupled to the system.

5. The system of claim 1, wherein the second element is configured to detect the another acceleration, the another acceleration being associated with the system.

6. The system of claim 1, wherein the acceleration is associated with a pulse.

7. The system of claim 1, wherein the acceleration is associated with a heart rate.

8. The system of claim 1, wherein the acceleration is associated with speech.

9. A system, comprising:
a first accelerometer configured to detect a first acceleration associated with a system element;
a second accelerometer configured to detect a second acceleration associated with the system; and
a differential amplifier configured to generate a signal corresponding to the first acceleration, wherein the signal is used to distinguish the first acceleration from the second acceleration.

10. A system, comprising:
an accelerometer configured to detect an acceleration, the accelerometer being coupled to a system element;
another accelerometer configured to detect another acceleration associated with the system; and
a differential amplifier configured to generate a signal corresponding to the acceleration, wherein the differential amplifier is configured to determine a difference between the acceleration and the another acceleration.

11. The system of claim 10, further comprising a matched coupling system configured to determine the difference between the acceleration and the another acceleration.

12. The system of claim 10, further comprising a coupling system configured to couple the accelerometer to the system element.

13. The system of claim 10, further comprising a coupling system configured to couple the another accelerometer to the system.
